(19) United States
(12) Patent Application Publication
(10) Pub. No.: US 2026/0137749 A1
(43) Pub. Date: May 21, 2026

(54) AUXILIARY LIPID-LOWERING CAMELLIA DIACYLGLYCEROL EDIBLE OIL AND PREPARATION METHOD THEREOF

(71) Applicant: Zishenghuo (Hunan) Food Research Institute Co., Ltd., Changsha (CN)

(72) Inventors: Boxing Chen, Changsha (CN); Lei Zhang, Changsha (CN)

(21) Appl. No.: 18/980,304

(22) Filed: Dec. 13, 2024

(30) Foreign Application Priority Data
Nov. 19, 2024 (CN) 2024116492620

(51) Int. Cl. A61K 36/82; A23D 9/013; A61K 36/39; A61K 36/46; A61K 36/54; A61K 36/70; A61K 36/899; A61K 47/18; A61K 47/36; A61K 47/44

(52) U.S. Cl. CPC A61K 36/82; A23D 9/013; A61K 36/39; A61K 36/46; A61K 36/54; A61K 36/70; A61K 36/899; A61K 47/183; A61K 47/36; A61K 47/44; A61K 2236/15; A61K 2236/33; A61K 2236/51

(57) ABSTRACT

An auxiliary lipid-lowering Camellia diacylglycerol edible oil and a preparation method thereof, involving grinding walnuts, peas, and sweet corn kernels, then thorough enzymatic hydrolysis; the obtained nutritious rapeseed oil is dispersed in the mixed edible oil, the prepared nutritious Camellia diacylglycerol oil mixture contains rich nutrients; the plant extract obtained by ultrasonic extraction is effectively adsorbed by the mixed colloid, the obtained nanoparticles that assist in lowering blood lipids and promoting metabolism cooperate with the nutritious Camellia diacylglycerol oil mixture to have an effective lipid-lowering effect; the antioxidant stabilizing composition and the nanoparticles that assist in lowering blood lipids and promoting metabolism are dispersed in the nutritious Camellia diacylglycerol oil mixture; the prepared auxiliary lipid-lowering Camellia diacylglycerol edible oil is rich in nutrition, does not increase blood lipids in the body after long-term consumption, doesn't cause damage to the liver and kidneys, and is not prone to deterioration.

### 1. TECHNICAL FIELD

The invention relates to the technical field of edible oil preparation, in particular to an auxiliary lipid-lowering Camellia diacylglycerol edible oil and a preparation method thereof.

### 2. BACKGROUND ART

Camellia diacylglycerol edible oil is a type of edible oil extracted from the seeds of Camellia oleifera belonging to the Theaceae family; it is a healthy edible oil prepared by converting triglycerides in ordinary edible oils into diglycerides; its primary component is diglyceride. Camellia diacylglycerol edible oil is rich in diglycerides and abundant unsaturated fatty acids such as linoleic acid and linolenic acid; it possesses heat resistance, making it suitable for high-temperature cooking.

The unsaturated fatty acid content in Camellia diacylglycerol edible oil exceeds that of olive oil, making it rank first among all edible oils; it can effectively eliminate free radicals in the body, slow down cellular aging, maintain youthful skin, promote intestinal motility, aid in digestion, and enhance the body's ability to absorb nutrients.

Currently, the existing edible oil preparation technologies have the following problems: first, long-term consumption of existing edible oils leads to elevated low-density lipoprotein cholesterol in the blood, which affects liver and kidney metabolism, reducing their ability to metabolize fats, and further increasing blood lipids; second, during the pressing process of existing edible oils, there is a loss of nutrients such as carotene, chlorophyll, phytosterols, and vitamin E, which results in a decrease in flavor and aroma; third, existing edible oils that have been opened for too long are prone to oxidation and rancidity in the presence of light, high temperature and air, with darker and deeper colors, turbidity and lower transparency, and the appearance of sediments and suspended matter.

### 3. SUMMARY OF THE INVENTION

In view of the above situation, in order to overcome the defects of the prior art, the invention provides an auxiliary lipid-lowering Camellia diacylglycerol edible oil and a preparation method thereof. In order to solve the problem that long-term consumption of existing edible oils leads to increased low-density lipoprotein cholesterol in the blood, damages liver and kidney metabolism, easy oxidation and rancidity, turbidity and reduced transparency during long-term storage, the invention uses a nutritious Camellia diacylglycerol oil mixture, nanoparticles that assist in lowering blood lipids and promoting metabolism, and an antioxidant stabilizing composition to prepare an auxiliary lipid-lowering Camellia diacylglycerol edible oil that is rich in nutrition, has a unique aroma, can repair damaged liver and kidneys, does not increase blood lipids in the body after long-term consumption, is not prone to deterioration during long-term storage, and has strong stability.

In order to realize the above objects, the invention adopts the following technical scheme: the invention proposes the auxiliary lipid-lowering Camellia diacylglycerol edible oil, preparation raw materials of the auxiliary lipid-lowering Camellia diacylglycerol edible oil specifically comprise the following components in parts by weight:
90-110 parts of nutritious Camellia diacylglycerol oil mixture, 30-44 parts of nanoparticles that assist in lowering blood lipids and promoting metabolism, 7-11 parts of bamboo leaf antioxidants, and 4-8 parts of ethylenediaminetetraacetic acid disodium salt.

Preferably, preparation raw materials of the nutritious Camellia diacylglycerol oil mixture comprise the following components in parts by weight: 10-12 parts of walnuts, 7-11 parts of peas, 6-9 parts of sweet corn kernels, 33-38 parts of rapeseed oil, 42-53 parts of Camellia diacylglycerol oil, 37-46 parts of Pinus koraiensis seed oil, 39-48 parts of sunflower seed oil, and 7-12 parts of soluble soybean polysaccharides; preparation raw materials of the nanoparticles that assist in lowering blood lipids and promoting metabolism comprise the following components in parts by weight: 14-18 parts of Semen cassiae, 16-19 parts of Radix Polygoni Multiflori, 4-9 parts of Cortex Eucommiae, 7-11 parts of Semen cuscutae, and 26-31 parts of sodium starch phosphate.

Preferably, a preparation method of the nutritious Camellia diacylglycerol oil mixture specifically comprises the following steps:
S1. putting walnuts, peas, and sweet corn kernels into a grinding machine with a power of 1.5-1.8 kW, the grinding speed is 3000 r/min, the grinding temperature is 28-33° C., and the grinding time is 10-15 minutes; after grinding, putting them, cellulose enzyme and bromelain into an enzymatic hydrolysis tank with a power of 1.8-2.2 kW, and the enzymatic hydrolysis temperature is 50-55° C., the hydrolysis time is 2-2.5 hours, and the hydrolysis speed is 70-90 r/min, stirring during hydrolysis to obtain a nutrient mixture;
S2. putting the nutrient mixture prepared in S1 and rapeseed oil into an ultrasonic extraction device with a power of 1.5 kW, the ultrasonic frequency is 20 kHz, the ultrasonic extraction temperature is 40° C., and the ultrasonic extraction time is 30 minutes; after ultrasonic extraction, placing the mixture to a high-speed centrifuge with a power of 1.6 kW, the centrifuge temperature is 2° C., the centrifuge speed is 22,000 r/min, and the centrifugation time is 15 minutes; after centrifugation, collecting the supernatant to obtain nutritious rapeseed oil;
S3. putting Camellia diacylglycerol oil, Pinus koraiensis seed oil and sunflower seed oil into a stirring tank with a power of 1.5-1.8 kW, stirring at a temperature of 25-28° C., a stirring speed of 130-150 r/min, and a stirring time of 15-25 min, thereby obtaining a mixed edible oil;
S4. placing the nutritious rapeseed oil prepared in S2, soluble soybean polysaccharides and the mixed edible oil prepared in S3 into a high-speed shear disperser with a power of 1.5-1.8 kW, and the high-speed shear dispersion temperature is 23-28° C., the high-speed shear dispersion time is 12-16 min, and the high-speed shear dispersion speed is 3000-4000 r/min, thereby obtaining a nutritious Camellia diacylglycerol oil mixture.

Preferably, a preparation method of the nanoparticles that assist in lowering blood lipids and promoting metabolism specifically comprises the following steps:
L1. putting Semen cassiae, Radix polygoni Multiflori, Cortex Eucommiae and Semen cuscutae into a pulverizer with a power of 1.4-1.8 kW, the pulverization temperature is 25-28° C., the pulverization time is 30-40 min, and the pulverization speed is 1200-1500 r/min; after pulverization, putting them and 70% ethanol solution into an ultrasonic extractor with a power of 1.5 kW, the ultrasonic frequency is 20 kHz, the ultrasonic extraction temperature is 40° C., and the ultrasonic extraction time is 30 min, thereby obtaining plant extract;
L2. putting the plant extract prepared in L1 and sodium starch phosphate into a stirring tank with a power of 1.2-1.4 kW, the stirring temperature is 50-60° C., the stirring speed is 120-140 r/min, the stirring time is 18-23 min; after stirring and mixing, placing it at a temperature of 25-28° C. for 1 h to obtain a mixed colloid;
L3. putting the mixed colloid prepared in L2 into a vacuum freeze-nano spray dryer with a power of 2.3-2.8 kW, with a vacuum degree of −0.05 MPa, a freeze-nano spray drying temperature of −40° C., a freeze-nano spray drying particle size of 100 nm, and a freeze-nano spray drying time of 2.5-3 h, thereby obtaining the nanoparticles that assist in lowering blood lipids and promoting metabolism.

Further, in step L1, the weight ratio of the 70% ethanol solution to Semen cassiae is 0.9-1.2:1.

This invention also provides a preparation method of the auxiliary lipid-lowering Camellia diacylglycerol edible oil, specifically comprising the following steps:
step 1: putting the bamboo leaf antioxidants and ethylenediaminetetraacetic acid disodium salt into a stirring tank with a power of 1.2-1.4 kW, stirring at a temperature of 20-26° C., a stirring speed of 150-180 r/min, and a stirring time of 8-12 min, thereby obtaining an antioxidant stabilizing composition;
step 2: adding the nanoparticles that assist in lowering blood lipids and promoting metabolism into the antioxidant stabilizing composition prepared in step 1, stirring at a temperature of 20-26° C., a stirring speed of 150-180 r/min, and a stirring time of 10-14 min, thereby obtaining a mixed preparation;
step 3: dispersing the mixed preparation prepared in step 2 in the nutritious Camellia diacylglycerol oil mixture, putting it into an ultrasonic stirrer with a power of 1.8 kW, an ultrasonic frequency of 15 kHz, a stirring speed of 20,000 r/min, a stirring temperature of 25° C., and a stirring time of 10 min, thereby obtaining the auxiliary lipid-lowering Camellia diacylglycerol edible oil.

The invention has the following advantages:
the invention involves grinding walnuts, peas, and sweet corn kernels into a pulp, followed by thorough enzymatic hydrolysis using cellulase and bromelain; the resulting nutrient mixture is then extracted by using rapeseed oil; after centrifugation, the supernatant is collected; the prepared nutritious rapeseed oil is rich in carotenoids, chlorophyll, phytosterols, and vitamin E; the blended edible oil contains Camellia diacylglycerol oil, Pinus koraiensis seed oil and sunflower seed oil; Camellia diacylglycerol oil is abundant in unsaturated fatty acids, Pinus koraiensis seed oil possesses a delicate fragrance of Pinus koraiensis seed and contains anthocyanins that can help lower blood lipids and protect cardiovascular and cerebrovascular health; sunflower seed oil has a refreshing aroma and contains a large amount of linoleic acid, which can reduce cholesterol accumulation in the blood; the nutritious rapeseed oil and soluble soybean polysaccharides are then dispersed in the blended edible oil through high-speed shearing; the soluble soybean polysaccharides increase dietary fiber intake and ensure stable dispersion of the nutritious rapeseed oil within the blended edible oil; the prepared nutritious Camellia diacylglycerol oil mixture has high nutritional value and stability; after crushing Semen cassiae, Radix polygoni Multiflori, Cortex Eucommiae and Semen cuscutae, they are extracted with a 70% ethanol solution; the resulting plant extract contains compounds such as obtusifolin, cassiaside, emodin, aucubin, coumarin, and hyperoside, and these compounds can help repair the liver, promote liver fat metabolism, inhibit cholesterol synthesis in the body, and convert it into bile acids, thereby enhancing the excretion of bile acids from the intestines, further reducing the absorption rate of fatty acids; the mixed colloid contains a three-dimensional network structure that effectively adsorbs the plant extract; after freeze-drying with a nano-spray technique, the resulting auxiliary lipid-lowering and metabolism-promoting nanoparticles have a small particle size and are evenly dispersed, working synergistically with the nutritious Camellia diacylglycerol oil mixture to achieve effective lipid-lowering effects; the antioxidant stabilizing composition is obtained by mixing bamboo leaf antioxidants and ethylenediaminetetraacetic acid disodium salt, it and the nanoparticles that assist in lowering blood lipids and promoting metabolism are dispersed in the nutritious Camellia diacylglycerol oil mixture; the prepared auxiliary lipid-lowering Camellia diacylglycerol edible oil is rich in nutrition, has a unique aroma, promotes the metabolic function of the liver and kidneys, can repair damaged liver and kidneys, does not increase blood lipids in the body after long-term consumption, is not prone to deterioration during long-term storage, and has strong stability.

### 4. BRIEF DESCRIPTION OF ACCOMPANY DRAWINGS

In order to explain the technical schemes in the embodiments of the invention or prior art more clearly, the accompanying drawings needed to be used in the description of the embodiments or the prior art will be briefly introduced below. Obviously, the accompanying drawings in the following description are only are some embodiments of the invention. For those of ordinary skill in the art, other accompanying drawings can be obtained based on these accompanying drawings without exerting creative efforts.

FIG. 1 is a scanning electron microscope image of the internal structure of the mixed colloid described in this invention.

FIG. 2 is a graph showing the results of blood lipid content in the hearts of rats from Experimental Example 1 of this invention.

FIG. 3 is a graph showing the results of the rat organ index from Experimental Example 1 of this invention.

FIG. 4 is a graph showing the transmittance results of Experimental Example 3 in the invention.

### 5. SPECIFIC EMBODIMENT OF THE INVENTION

The technical schemes in the embodiments of the invention will be clearly and completely described in combination with the accompanying drawings in the embodiments of the invention. Obviously, the described embodiments are only some of the embodiments of the invention, but not all of the embodiments. Based on the embodiments in this invention, all other embodiments obtained by those of ordinary skill in the art without making creative efforts shall fall within the protection scope of this invention.

Unless otherwise defined, all professional and scientific terms used herein have the same meanings as those familiar to those skilled in the art. In addition, any methods and materials similar or equivalent to those described herein can be applied to the invention. The preferred embodiments and materials described herein are for demonstration purposes only and do not limit the content of the invention.

The experimental methods in the following embodiments are all conventional methods unless otherwise specified; the experimental instruments and materials used in the following embodiments are all purchased from commercial channels unless otherwise specified.

Embodiment 1: this embodiment provides an auxiliary lipid-lowering Camellia diacylglycerol edible oil and a preparation method thereof; preparation raw materials of the auxiliary lipid-lowering Camellia diacylglycerol edible oil specifically comprise the following components in parts by weight:
90 parts of nutritious Camellia diacylglycerol oil mixture, 30 parts of nanoparticles that assist in lowering blood lipids and promoting metabolism, 7 parts of bamboo leaf antioxidants, and 4 parts of ethylenediaminetetraacetic acid disodium salt.

Preparation raw materials of the nutritious Camellia diacylglycerol oil mixture comprise the following components in parts by weight: 10 parts of walnuts, 7 parts of peas, 6 parts of sweet corn kernels, 33 parts of rapeseed oil, 42 parts of Camellia diacylglycerol oil, 37 parts of Pinus koraiensis seed oil, 39 parts of sunflower seed oil, and 7 parts of soluble soybean polysaccharides; preparation raw materials of the nanoparticles that assist in lowering blood lipids and promoting metabolism comprise the following components in parts by weight: 14 parts of Semen cassiae, 16 parts of Radix polygoni Multiflori, 4 parts of Cortex Eucommiae, 7 parts of Semen cuscutae, and 26 parts of sodium starch phosphate.

A preparation method of the nutritious Camellia diacylglycerol oil mixture specifically comprises the following steps:
S1. putting walnuts, peas, and sweet corn kernels into a grinding machine with a power of 1.5 kW, the grinding speed is 3000 r/min, the grinding temperature is 28° C., and the grinding time is 10 minutes; after grinding, putting them, cellulose enzyme and bromelain into an enzymatic hydrolysis tank with a power of 1.8 kW, and the enzymatic hydrolysis temperature is 50° C., the hydrolysis time is 2 hours, and the hydrolysis speed is 70 r/min, stirring during hydrolysis to obtain a nutrient mixture;
S2. putting the nutrient mixture prepared in S1 and rapeseed oil into an ultrasonic extraction device with a power of 1.5 kW, the ultrasonic frequency is 20 kHz, the ultrasonic extraction temperature is 40° C., and the ultrasonic extraction time is 30 minutes; after ultrasonic extraction, placing the mixture to a high-speed centrifuge with a power of 1.6 kW, the centrifuge temperature is 2° C., the centrifuge speed is 22,000 r/min, and the centrifugation time is 15 minutes; after centrifugation, collecting the supernatant to obtain nutritious rapeseed oil;
S3. putting Camellia diacylglycerol oil, Pinus koraiensis seed oil and sunflower seed oil into a stirring tank with a power of 1.5 kW, stirring at a temperature of 25° C., a stirring speed of 130 r/min, and a stirring time of 15 min, thereby obtaining a mixed edible oil;
S4. placing the nutritious rapeseed oil prepared in S2, soluble soybean polysaccharides and the mixed edible oil prepared in S3 into a high-speed shear disperser with a power of 1.5 kW, and the high-speed shear dispersion temperature is 23° C., the high-speed shear dispersion time is 12 min, and the high-speed shear dispersion speed is 3000 r/min, thereby obtaining a nutritious Camellia diacylglycerol oil mixture.

A preparation method of the nanoparticles that assist in lowering blood lipids and promoting metabolism specifically comprises the following steps:
L1. putting Semen cassiae, Radix polygoni Multiflori, Cortex Eucommiae and Semen cuscutae into a pulverizer with a power of 1.4 kW, the pulverization temperature is 25° C., the pulverization time is 30 min, and the pulverization speed is 1200 r/min; after pulverization, putting them and 70% ethanol solution into an ultrasonic extractor with a power of 1.5 kW, the ultrasonic frequency is 20 kHz, the ultrasonic extraction temperature is 40° C., and the ultrasonic extraction time is 30 min, thereby obtaining plant extract;
L2. putting the plant extract prepared in L1 and sodium starch phosphate into a stirring tank with a power of 1.2 kW, the stirring temperature is 50° C., the stirring speed is 120 r/min, the stirring time is 18 min; after stirring and mixing, placing it at a temperature of 25° C. for 1 h to obtain a mixed colloid;
L3. putting the mixed colloid prepared in L2 into a vacuum freeze-nano spray dryer with a power of 2.3 kW, with a vacuum degree of −0.05 MPa, a freeze-nano spray drying temperature of −40° C., a freeze-nano spray drying particle size of 100 nm, and a freeze-nano spray drying time of 2.5 h, thereby obtaining the nanoparticles that assist in lowering blood lipids and promoting metabolism.

In step L1, the weight ratio of the 70% ethanol solution to Semen cassiae is 0.9:1.

This embodiment also provides a preparation method of the auxiliary lipid-lowering Camellia diacylglycerol edible oil, specifically comprising the following steps:
step 1: putting the bamboo leaf antioxidants and ethylenediaminetetraacetic acid disodium salt into a stirring tank with a power of 1.2 kW, stirring at a temperature of 20° C., a stirring speed of 150 r/min, and a stirring time of 8 min, thereby obtaining an antioxidant stabilizing composition;
step 2: adding the nanoparticles that assist in lowering blood lipids and promoting metabolism into the antioxidant stabilizing composition prepared in step 1, stirring at a temperature of 20° C., a stirring speed of 150 r/min, and a stirring time of 10 min, thereby obtaining a mixed preparation;
step 3: dispersing the mixed preparation prepared in step 2 in the nutritious Camellia diacylglycerol oil mixture, putting it into an ultrasonic stirrer with a power of 1.8 kW, an ultrasonic frequency of 15 kHz, a stirring speed of 20,000 r/min, a stirring temperature of 25° C., and a stirring time of 10 min, thereby obtaining the auxiliary lipid-lowering Camellia diacylglycerol edible oil.

Embodiment 2: this embodiment provides an auxiliary lipid-lowering Camellia diacylglycerol edible oil and a preparation method thereof; preparation raw materials of the auxiliary lipid-lowering Camellia diacylglycerol edible oil specifically comprise the following components in parts by weight:
100 parts of nutritious Camellia diacylglycerol oil mixture, 38 parts of nanoparticles that assist in lowering blood lipids and promoting metabolism, 9 parts of bamboo leaf antioxidants, and 7 parts of ethylenediaminetetraacetic acid disodium salt.

Preparation raw materials of the nutritious Camellia diacylglycerol oil mixture comprise the following components in parts by weight: 11 parts of walnuts, 9 parts of peas, 8 parts of sweet corn kernels, 36 parts of rapeseed oil, 48 parts of Camellia diacylglycerol oil, 41 parts of Pinus koraiensis seed oil, 42 parts of sunflower seed oil, and 10 parts of soluble soybean polysaccharides; preparation raw materials of the nanoparticles that assist in lowering blood lipids and promoting metabolism comprise the following components in parts by weight: 16 parts of Semen cassiae, 18 parts of Radix polygoni Multiflori, 6 parts of Cortex Eucommiae, 10 parts of Semen cuscutae, and 29 parts of sodium starch phosphate.

A preparation method of the nutritious Camellia diacylglycerol oil mixture specifically comprises the following steps:
S1. putting walnuts, peas, and sweet corn kernels into a grinding machine with a power of 1.6 kW, the grinding speed is 3000 r/min, the grinding temperature is 30° C., and the grinding time is 13 minutes; after grinding, putting them, cellulose enzyme and bromelain into an enzymatic hydrolysis tank with a power of 2.1 kW, and the enzymatic hydrolysis temperature is 52° C., the hydrolysis time is 2.3 hours, and the hydrolysis speed is 80 r/min, stirring during hydrolysis to obtain a nutrient mixture;
S2. putting the nutrient mixture prepared in S1 and rapeseed oil into an ultrasonic extraction device with a power of 1.5 kW, the ultrasonic frequency is 20 kHz, the ultrasonic extraction temperature is 40° C., and the ultrasonic extraction time is 30 minutes; after ultrasonic extraction, placing the mixture to a high-speed centrifuge with a power of 1.6 kW, the centrifuge temperature is 2° C., the centrifuge speed is 22,000 r/min, and the centrifugation time is 15 minutes; after centrifugation, collecting the supernatant to obtain nutritious rapeseed oil;
S3. putting Camellia diacylglycerol oil, Pinus koraiensis seed oil and sunflower seed oil into a stirring tank with a power of 1.6 kW, stirring at a temperature of 27° C., a stirring speed of 140 r/min, and a stirring time of 20 min, thereby obtaining a mixed edible oil;
S4. placing the nutritious rapeseed oil prepared in S2, soluble soybean polysaccharides and the mixed edible oil prepared in S3 into a high-speed shear disperser with a power of 1.6 kW, and the high-speed shear dispersion temperature is 25° C., the high-speed shear dispersion time is 14 min, and the high-speed shear dispersion speed is 3000 r/min, thereby obtaining a nutritious Camellia diacylglycerol oil mixture.

A preparation method of the nanoparticles that assist in lowering blood lipids and promoting metabolism specifically comprises the following steps:
L1. putting Semen cassiae, Radix polygoni Multiflori, Cortex Eucommiae and Semen cuscutae into a pulverizer with a power of 1.7 kW, the pulverization temperature is 26° C., the pulverization time is 35 min, and the pulverization speed is 1300 r/min; after pulverization, putting them and 70% ethanol solution into an ultrasonic extractor with a power of 1.5 kW, the ultrasonic frequency is 20 kHz, the ultrasonic extraction temperature is 40° C., and the ultrasonic extraction time is 30 min, thereby obtaining plant extract;
L2. putting the plant extract prepared in L1 and sodium starch phosphate into a stirring tank with a power of 1.3 kW, the stirring temperature is 55° C., the stirring speed is 130 r/min, the stirring time is 22 min; after stirring and mixing, placing it at a temperature of 26° C. for 1 h to obtain a mixed colloid;
L3. putting the mixed colloid prepared in L2 into a vacuum freeze-nano spray dryer with a power of 2.5 kW, with a vacuum degree of −0.05 MPa, a freeze-nano spray drying temperature of −40° C., a freeze-nano spray drying particle size of 100 nm, and a freeze-nano spray drying time of 2.8 h, thereby obtaining the nanoparticles that assist in lowering blood lipids and promoting metabolism.

In step L1, the weight ratio of the 70% ethanol solution to Semen cassiae is 1.1:1.

This embodiment also provides a preparation method of the auxiliary lipid-lowering Camellia diacylglycerol edible oil, specifically comprising the following steps:
step 1: putting the bamboo leaf antioxidants and ethylenediaminetetraacetic acid disodium salt into a stirring tank with a power of 1.3 kW, stirring at a temperature of 23° C., a stirring speed of 160 r/min, and a stirring time of 10 min, thereby obtaining an antioxidant stabilizing composition;
step 2: adding the nanoparticles that assist in lowering blood lipids and promoting metabolism into the antioxidant stabilizing composition prepared in step 1, stirring at a temperature of 22° C., a stirring speed of 170 r/min, and a stirring time of 12 min, thereby obtaining a mixed preparation;
step 3: dispersing the mixed preparation prepared in step 2 in the nutritious Camellia diacylglycerol oil mixture, putting it into an ultrasonic stirrer with a power of 1.8 kW, an ultrasonic frequency of 15 kHz, a stirring speed of 20,000 r/min, a stirring temperature of 25° C., and a stirring time of 10 min, thereby obtaining the auxiliary lipid-lowering Camellia diacylglycerol edible oil.

Embodiment 3: this embodiment provides an auxiliary lipid-lowering Camellia diacylglycerol edible oil and a preparation method thereof; preparation raw materials of the auxiliary lipid-lowering Camellia diacylglycerol edible oil specifically comprise the following components in parts by weight:
110 parts of nutritious Camellia diacylglycerol oil mixture, 44 parts of nanoparticles that assist in lowering blood lipids and promoting metabolism, 11 parts of bamboo leaf antioxidants, and 8 parts of ethylenediaminetetraacetic acid disodium salt.

Preparation raw materials of the nutritious Camellia diacylglycerol oil mixture comprise the following components in parts by weight: 12 parts of walnuts, 11 parts of peas, 9 parts of sweet corn kernels, 38 parts of rapeseed oil, 53 parts of Camellia diacylglycerol oil, 46 parts of Pinus koraiensis seed oil, 48 parts of sunflower seed oil, and 12 parts of soluble soybean polysaccharides; preparation raw materials of the nanoparticles that assist in lowering blood lipids and promoting metabolism comprise the following components in parts by weight: 18 parts of Semen cassiae, 19 parts of Radix polygoni Multiflori, 9 parts of Cortex Eucommiae, 11 parts of Semen cuscutae, and 31 parts of sodium starch phosphate.

A preparation method of the nutritious Camellia diacylglycerol oil mixture specifically comprises the following steps:
S1. putting walnuts, peas, and sweet corn kernels into a grinding machine with a power of 1.8 KW, the grinding speed is 3000 r/min, the grinding temperature is 33° C., and the grinding time is 15 minutes; after grinding, putting them, cellulose enzyme and bromelain into an enzymatic hydrolysis tank with a power of 2.2 kW, and the enzymatic hydrolysis temperature is 55° C., the hydrolysis time is 2.5 hours, and the hydrolysis speed is 90 r/min, stirring during hydrolysis to obtain a nutrient mixture;
S2. putting the nutrient mixture prepared in S1 and rapeseed oil into an ultrasonic extraction device with a power of 1.5 kW, the ultrasonic frequency is 20 kHz, the ultrasonic extraction temperature is 40° C., and the ultrasonic extraction time is 30 minutes; after ultrasonic extraction, placing the mixture to a high-speed centrifuge with a power of 1.6 kW, the centrifuge temperature is 2° C., the centrifuge speed is 22,000 r/min, and the centrifugation time is 15 minutes; after centrifugation, collecting the supernatant to obtain nutritious rapeseed oil;
S3. putting Camellia diacylglycerol oil, Pinus koraiensis seed oil and sunflower seed oil into a stirring tank with a power of 1.8 kW, stirring at a temperature of 28° C., a stirring speed of 150 r/min, and a stirring time of 25 min, thereby obtaining a mixed edible oil;
S4. placing the nutritious rapeseed oil prepared in S2, soluble soybean polysaccharides and the mixed edible oil prepared in S3 into a high-speed shear disperser with a power of 1.8 kW, and the high-speed shear dispersion temperature is 28° C., the high-speed shear dispersion time is 16 min, and the high-speed shear dispersion speed is 4000 r/min, thereby obtaining a nutritious Camellia diacylglycerol oil mixture.

A preparation method of the nanoparticles that assist in lowering blood lipids and promoting metabolism specifically comprises the following steps:
L1. putting Semen cassiae, Radix polygoni Multiflori, Cortex Eucommiae and Semen cuscutae into a pulverizer with a power of 1.8 kW, the pulverization temperature is 28° C., the pulverization time is 40 min, and the pulverization speed is 1500 r/min; after pulverization, putting them and 70% ethanol solution into an ultrasonic extractor with a power of 1.5 kW, the ultrasonic frequency is 20 kHz, the ultrasonic extraction temperature is 40° C., and the ultrasonic extraction time is 30 min, thereby obtaining plant extract;
L2. putting the plant extract prepared in L1 and sodium starch phosphate into a stirring tank with a power of 1.4 kW, the stirring temperature is 60° C., the stirring speed is 140 r/min, the stirring time is 23 min; after stirring and mixing, placing it at a temperature of 28° C. for 1 h to obtain a mixed colloid;
L3. putting the mixed colloid prepared in L2 into a vacuum freeze-nano spray dryer with a power of 2.8 kW, with a vacuum degree of −0.05 MPa, a freeze-nano spray drying temperature of −40° C., a freeze-nano spray drying particle size of 100 nm, and a freeze-nano spray drying time of 3 h, thereby obtaining the nanoparticles that assist in lowering blood lipids and promoting metabolism.

In step L1, the weight ratio of the 70% ethanol solution to Semen cassiae is 1.2:1.

This embodiment also provides a preparation method of the auxiliary lipid-lowering Camellia diacylglycerol edible oil, specifically comprising the following steps:
step 1: putting the bamboo leaf antioxidants and ethylenediaminetetraacetic acid disodium salt into a stirring tank with a power of 1.4 kW, stirring at a temperature of 26° C., a stirring speed of 180 r/min, and a stirring time of 12 min, thereby obtaining an antioxidant stabilizing composition;
step 2: adding the nanoparticles that assist in lowering blood lipids and promoting metabolism into the antioxidant stabilizing composition prepared in step 1, stirring at a temperature of 26° C., a stirring speed of 180 r/min, and a stirring time of 14 min, thereby obtaining a mixed preparation;
step 3: dispersing the mixed preparation prepared in step 2 in the nutritious Camellia diacylglycerol oil mixture, putting it into an ultrasonic stirrer with a power of 1.8 kW, an ultrasonic frequency of 15 kHz, a stirring speed of 20,000 r/min, a stirring temperature of 25° C., and a stirring time of 10 min, thereby obtaining the auxiliary lipid-lowering Camellia diacylglycerol edible oil.

Comparative Example 1: this comparative example provides a Camellia diacylglycerol edible oil and a preparation method thereof, which is different from Embodiment 1 only in that the added nutritious Camellia diacylglycerol oil mixture does not contain a nutrient mixture, the remaining components, component contents, steps are the same as those in Embodiment 1.

Comparative Example 2: this comparative example provides a Camellia diacylglycerol edible oil and a preparation method thereof, which is different from Embodiment 1 only in that the added auxiliary lipid-lowering and metabolic promoting agent does not contain plant extracts, the remaining components, component contents, steps are the same as those in Embodiment 1.

### Experimental Example 1: Measurement of Lipid-Lowering Effect and Impact on Liver and Kidney

The steps for testing the lipid-lowering effect and the impact on the liver and kidneys of the auxiliary lipid-lowering Camellia diacylglycerol edible oil prepared in Embodiments 1-3 of the invention are as follows:

(1) selecting 60 male SD rats, each weighing 220 g (purchased from Chengdu Dashuo Animal Co., Ltd.), and grouping them in sets of 10 rats per group, randomly dividing the rats into 6 groups, with each group housed in separate cages; maintaining the cages in a clean and dry condition, with a temperature of 24° C., humidity of 50%, and free access to food and water for feeding the rats;

(2) after 10 days of feeding, the rats in Embodiment 1-3 groups were fed with 2.0 mL of the auxiliary lipid-lowering Camellia diacylglycerol edible oil prepared in Embodiment 1-3, the rats in Comparative Example 1-2 groups were fed with 2.0 mL of the Camellia diacylglycerol edible oil prepared in Comparative Example 1-2, and the rats in the control group were fed with 2.0 mL of commercially available ordinary Camellia oil (purchased from Zhongshan Yaofu Food Co., Ltd.), three times a day, for 13 weeks;

(3) after being fed for 13 weeks, the rats in each group were fasted for 24 h and killed by cervical dislocation; the rat heart blood was collected, and the blood was kept in a water bath at 37° C. for 5 min and centrifuged at 4000 r/min for 5 min; then the supernatant was collected and the levels of triglyceride and low-density lipoprotein cholesterol in serum were detected by an automatic biochemical analyzer and recorded; the obtained data were statistically analyzed by using IBM SPSS Statistics 22.0 software, and the graphics were drawn by using Origin2022;

(4) the liver and kidney of each rat were separated, rinsed with 0.9% saline, and then the surface moisture was absorbed with filter paper and weighed quickly; the organ index (%)=weight of a certain organ/rat body weight×100%; an increase in the organ index indicates congestion, edema, or hyperplasia of the organ, and a decrease in the organ index indicates organ atrophy and other degenerative changes.

### Results Analysis:

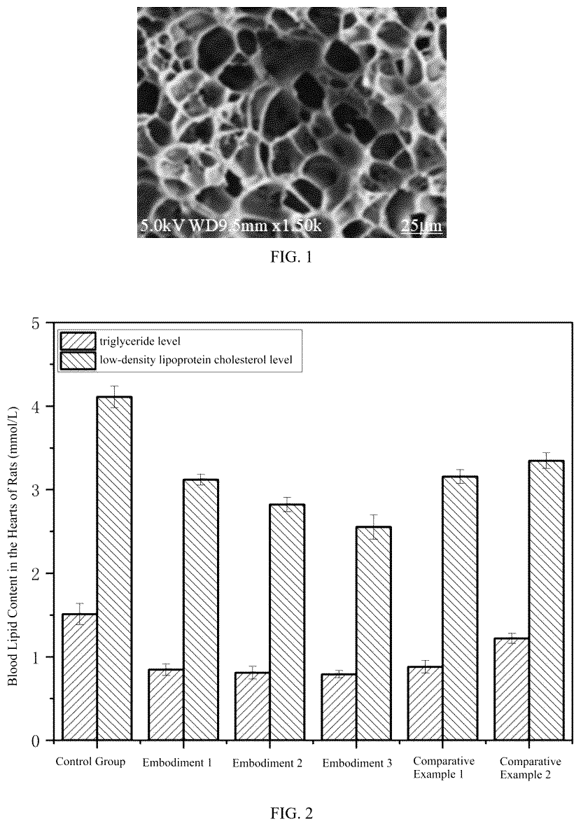

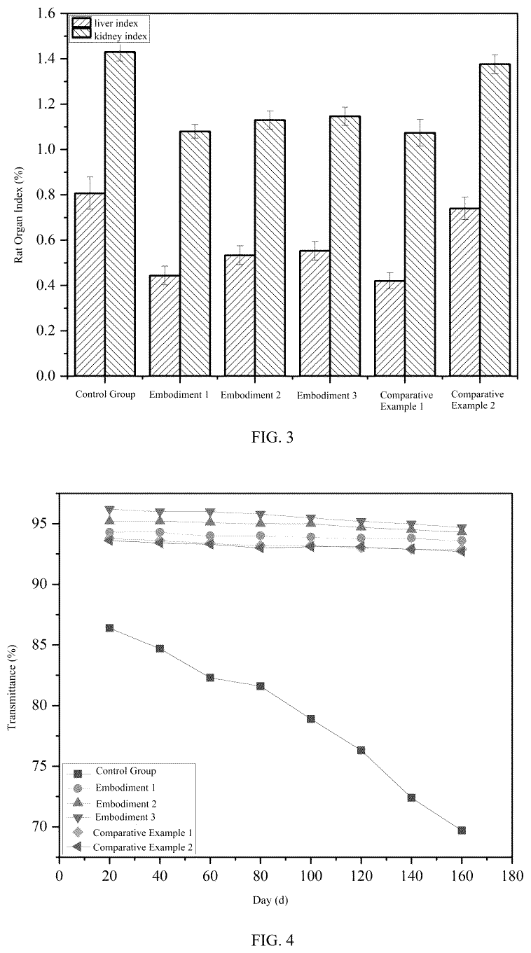

FIG. 1 is a scanning electron microscope image of the internal structure of the mixed colloid described in this invention. FIG. 2 is a graph showing the results of blood lipid content in the hearts of rats from Experimental Example 1 of this invention. As shown in the figure, rats in Embodiment 1-3 groups were fed with 2.0 mL of the auxiliary lipid-lowering Camellia diacylglycerol edible oil prepared in Embodiment 1-3, respectively, three times a day for 13 consecutive weeks; after this feeding period, the triglyceride levels in the serum of rats in Embodiment 1-3 groups were 0.85 mmol/L, 0.81 mmol/L, and 0.79 mmol/L, respectively, and the low-density lipoprotein cholesterol levels in their serum were 3.12 mmol/L, 2.82 mmol/L, and 2.55 mmol/L, respectively. Rats in Comparative Example 1 Group were fed with 2.0 mL of the Camellia diacylglycerol edible oil prepared in Comparative Example 1, three times a day for 13 consecutive weeks; after this period, the triglyceride levels in their serum were 0.88 mmol/L, and the low-density lipoprotein cholesterol levels were 3.16 mmol/L. Rats in Comparative Example 2 Group were fed with 2.0 mL of the Camellia diacylglycerol edible oil prepared in Comparative Example 2, three times a day for 13 consecutive weeks; after this period, the triglyceride levels in their serum were 1.22 mmol/L, and the low-density lipoprotein cholesterol levels were 3.35 mmol/L. Rats in the control group were fed with 2.0 mL of commercially available ordinary Camellia oil, three times a day for 13 consecutive weeks; after this feeding period, the triglyceride levels in their serum were 1.51 mmol/L, and the low-density lipoprotein cholesterol levels were 4.11 mmol/L. FIG. 3 is a graph showing the results of the rat organ index from Experimental Example 1 of this invention. As shown in the figure, rats in Embodiment 1-3 groups were fed with 2.0 mL of the auxiliary lipid-lowering Camellia diacylglycerol edible oil prepared in Embodiment 1-3, respectively, three times a day for 13 consecutive weeks; after this feeding period, the liver indices of the rats were 0.44%, 0.53%, and 0.55%, respectively, while the kidney indices were 1.08%, 1.13%, and 1.15%, respectively. Rats in Comparative Example 1 Group were fed with 2.0 mL of the Camellia diacylglycerol edible oil prepared in Comparative Example 1, three times a day for 13 consecutive weeks; after this period, the liver index was 0.42% and the kidney index was 1.07%. Rats in Comparative Example 2 Group were fed with 2.0 mL of the Camellia diacylglycerol edible oil prepared in Comparative Example 2, three times a day for 13 consecutive weeks; after this period, the liver index was 0.74% and the kidney index was 1.38%. Rats in the control group were fed with 2.0 mL of commercially available ordinary Camellia oil, three times a day for 13 consecutive weeks; after this feeding period, the liver index was 0.81% and the kidney index was 1.43%. These results indicate that long-term consumption of the auxiliary lipid-lowering Camellia diacylglycerol edible oil prepared in this invention does not elevate blood lipids in the body, has a repairing effect on the liver and kidneys, without affecting the normal metabolism of fat by liver and kidneys.

### Experimental Example 2: Nutrient Content Measurement Test

The steps for measuring the nutrient content of the auxiliary lipid-lowering Camellia diacylglycerol edible oil prepared in Embodiments 1-3 of the invention are as follows:
(1) the method for determining the carotene content in each group of edible oils refers to the standard GB/T 5009.83-2016 “Determination of Carotene in Foods.” The method for determining the chlorophyll content refers to the standard NY/T 3082-2017 “Determination of Chlorophyll Content in Fruits, Vegetables, and Their Products.” The method for determining the phytosterol content refers to the standard GB/T 39995-2021 “Determination of Sterols.” The method for determining the vitamin E content refers to the standard GB5009.82-2016 “Determination of Vitamins A, D, and E in Foods;”
(2) 1 L of the auxiliary lipid-lowering Camellia diacylglycerol edible oil prepared in Embodiments 1-3 was taken as Embodiments 1-3 groups, 1 L of the Camellia diacylglycerol edible oil prepared in Comparative Example 1-2 was taken as Comparative Example 1-2 groups, and 1 L of commercially available ordinary Camellia oil (purchased from Zhongshan Yaofu Food Co., Ltd.) was taken as the control group. Five parallel measurements were set for each group, and the average value was taken. The results are recorded in Table 1.

TABLE 1
Nutritional Content Determination Table of Edible Oil

| | Carotene | Chlorophyll | Phytosterol | Vitamin E |
|---|---|---|---|---|
| | (mg/L) | (mg/L) | (mg/L) | (mg/L) |
| Control Group | 20.44 | 15.71 | 652.25 | 65.72 |
| Embodiment 1 | 44.23 | 31.68 | 716.39 | 91.37 |
| Embodiment 2 | 47.33 | 33.45 | 724.87 | 98.74 |
| Embodiment 3 | 49.87 | 37.12 | 739.64 | 101.23 |
| Comparative | 23.15 | 18.47 | 660.03 | 70.31 |
| Example 1 | | | | |
| Comparative | 42.78 | 29.87 | 711.82 | 90.85 |
| Example 2 | | | | |

### Result Analysis:

As shown in Table 1, the carotene, chlorophyll, phytosterol, and vitamin E content in the auxiliary lipid-lowering Camellia diacylglycerol edible oil prepared in Embodiments 1-3 are higher than those in the Camellia diacylglycerol edible oil prepared in Comparative Example 1 and the commercially available ordinary Camellia oil. This indicates that the use of the nutritional mixture compensates for the loss of nutrients such as carotene, chlorophyll, phytosterol, and vitamin E during the edible oil extraction process; after high-speed shearing and dispersion, the nutritious rapeseed oil can exist stably in mixed edible oil for a long time.

### Experimental Example 3: Long-Term Storage Stability Test

The steps for determining the long-term storage stability of the auxiliary lipid-lowering Camellia diacylglycerol edible oil prepared in Embodiments 1-3 of the invention are as follows:
(1) taking 100 mL of the auxiliary lipid-lowering Camellia diacylglycerol edible oil prepared in Embodiments 1-3 as samples for the Embodiment 1-3 groups, respectively; taking 100 mL of the Camellia diacylglycerol edible oil prepared in Comparative Examples 1-2 as samples for the Comparative Example 1-2 groups; taking 100 mL of commercially available ordinary Camellia oil (purchased from Zhongshan Yaofu Food Co., Ltd.) as samples for the control group; six parallels are taken for each group;
(2) storing the samples in the dark at 20° C.; on the 120th day of storage, taking out the edible oil samples from each group, evaluating and scoring their color, appearance, odor, and taste according to the scoring criteria in Table 2, taking the average value for recording, and the results are shown in Table 3;
(3) storing the edible oil samples from each group in the dark at 20° C.; on the 20th, 40th, 60th, 80th, 100th, 120th, 140th, and 160th days of storage, taking out the samples from each group and letting them stand for 10 minutes; then, using an LS116 transmittance meter to measure and record their transmittance.

TABLE 2
Edible Oil Sample Scoring Standard Table

| Evaluation | Scoring Criteria/Points | | |
|---|---|---|---|
| Indicators | 1 | 3 | 5 |
| color and | turbid with | turbid without | turbid without |
| appearance | sediment | sediment | sediment |
| odor | weak aroma | average aroma | obvious aroma |
| taste | sour and greasy | sour and greasy | fragrant and |
| | | | delicate |

TABLE 3
Edible Oil Sample Scoring Table

| Scoring | Control | Embodiment | Embodiment | Embodiment | Comparative | Comparative |
|---|---|---|---|---|---|---|
| Criteria | Group | 1 | 2 | 3 | Example 1 | Example 2 |
| color | 3.47 | 5.00 | 5.00 | 5.00 | 5.00 | 5.00 |
| odor | 3.26 | 5.00 | 5.00 | 5.00 | 5.00 | 5.00 |
| taste | 3.32 | 5.00 | 5.00 | 5.00 | 5.00 | 5.00 |

### Result Analysis:

FIG. 4 is a graph showing the transmittance results of Experimental Example 3 in the invention. As shown in Table 3 and FIG. 4, the auxiliary lipid-lowering Camellia diacylglycerol edible oil prepared in Embodiments 1-3, and the Camellia diacylglycerol edible oil prepared in Comparative Examples 1-2, were stored in the dark at 20° C.; after the 120th day of storage, their color, appearance, odor, and taste scores were higher than those of the commercially available ordinary Camellia oil. Moreover, the auxiliary lipid-lowering Camellia diacylglycerol edible oil prepared in Embodiment 1 was stored at 20° C. in the dark; at the 20th, 40th, 60th, 80th, 100th, 120th, 140th, and 160th day of storage, the transmittance of each group of samples after being taken out and left for 10 minutes was measured to be 94.3%, 94.3%, 94.0%, 94.0%, 93.9%, 93.8%, 93.8%, and 93.6%, respectively. The Camellia diacylglycerol edible oil prepared in Comparative Example 1 was stored at 20° C. in the dark; at the 20th, 40th, 60th, 80th, 100th, 120th, 140th, and 160th day of storage, the transmittance of each group of samples after being taken out and left for 10 minutes was measured to be 93.8%, 93.6%, 93.4%, 93.2%, 93.2%, 93.0%, 92.9%, and 92.9%. The Camellia diacylglycerol edible oil prepared in Comparative Example 2 was stored at 20° C. in the dark; at the 20th, 40th, 60th, 80th, 100th, 120th, 140th, and 160th day of storage, the transmittance of each group of samples after being taken out and left for 10 minutes was measured to be 93.6%, 93.4%, 93.3%, 93.0%, 93.1%, 93.1%, 92.9%, and 92.7%. The commercially available ordinary Camellia oil was stored at 20° C. in the dark; at the 20th, 40th, 60th, 80th, 100th, 120th, 140th, and 160th day of storage, the transmittance of each group of samples after being taken out and left for 10 minutes was measured to be 86.4%, 84.7%, 82.3%, 81.6%, 78.9%, 76.3%, 72.4%, and 69.7%. This demonstrates that the auxiliary lipid-lowering Camellia diacylglycerol edible oil prepared by this invention has a unique aroma, good taste, and strong stability during long-term storage, without precipitation, and maintains high clarity.

Although the embodiments of the invention have been shown and described above, it should be understood that those skilled in the art can make changes, modifications, substitutions and alterations to the above embodiments without departing from the principles and spirit of the invention. The scope of the invention is defined by the claims and their equivalents.

The invention and its embodiments are described above, this description is not restrictive, and what is shown in the accompanying drawing is only one of the embodiments of the invention, and the actual structure is not limited to this. All in all, if those skilled in the art receives its enlightenment, without deviating from the object of the invention, and without creatively designing structures and embodiments similar to the technical scheme of the invention shall fall within the protection scope of the invention.

1. An auxiliary lipid-lowering Camellia diacylglycerol edible oil, wherein preparation raw materials of the auxiliary lipid-lowering Camellia diacylglycerol edible oil specifically comprise the following components in parts by weight:
90-110 parts of nutritious Camellia diacylglycerol oil mixture, 30-44 parts of nanoparticles that assist in lowering blood lipids and promoting metabolism, 7-11 parts of bamboo leaf antioxidants, and 4-8 parts of ethylenediaminetetraacetic acid disodium salt;
preparation raw materials of the nutritious Camellia diacylglycerol oil mixture comprise the following components in parts by weight: 10-12 parts of walnuts, 7-11 parts of peas, 6-9 parts of sweet corn kernels, 33-38 parts of rapeseed oil, 42-53 parts of Camellia diacylglycerol oil, 37-46 parts of Pinus koraiensis seed oil, 39-48 parts of sunflower seed oil, and 7-12 parts of soluble soybean polysaccharides;
preparation raw materials of the nanoparticles that assist in lowering blood lipids and promoting metabolism comprise the following components in parts by weight: 14-18 parts of Semen cassiae, 16-19 parts of Radix polygoni Multiflori, 4-9 parts of Cortex Eucommiae, 7-11 parts of Semen cuscutae, and 26-31 parts of sodium starch phosphate.

2. The auxiliary lipid-lowering Camellia diacylglycerol edible oil of claim 1, wherein a preparation method of the nutritious Camellia diacylglycerol oil mixture specifically comprises the following steps:
S1: grinding walnuts, peas and sweet corn kernels and performing enzymatic hydrolysis to obtain a nutrient mixture;
S2: performing ultrasonic extraction on the nutrient mixture prepared in S1 through rapeseed oil, and then performing centrifugation to obtain supernatant, thereby obtaining nutritious rapeseed oil;
S3: stirring and mixing Camellia diacylglycerol oil, Pinus koraiensis seed oil and sunflower seed oil to obtain a mixed edible oil;
S4: performing high-speed shear dispersion on the nutritious rapeseed oil prepared in S2, soluble soybean polysaccharides and the mixed edible oil prepared in S3 to obtain a nutritious Camellia diacylglycerol oil mixture.

3. The auxiliary lipid-lowering Camellia diacylglycerol edible oil of claim 2, wherein a preparation method of the nanoparticles that assist in lowering blood lipids and promoting metabolism specifically comprises the following steps:
L1: mixing and crushing Semen cassiae, Radix polygoni Multiflori, Cortex Eucommiae and Semen cuscutae, and using 70% ethanol solution for ultrasonic extraction to obtain plant extract;
L2: mixing the plant extract prepared in L1 with sodium starch phosphate, and placing it at a temperature of 25-28° C. for 1 hour to obtain a mixed colloid;
L3: performing freeze-nano spray drying on the mixed colloid prepared in L2 to obtain the nanoparticles that assist in lowering blood lipids and promoting metabolism.

4. The auxiliary lipid-lowering Camellia diacylglycerol edible oil of claim 3, wherein in step L1, the weight ratio of the 70% ethanol solution to Semen cassiae is 0.9-1.2:1.

5. A preparation method of the auxiliary lipid-lowering Camellia diacylglycerol edible oil of claim 1, specifically comprising the following steps:
step 1: stirring and mixing bamboo leaf antioxidants and ethylenediaminetetraacetic acid disodium salt to obtain an antioxidant stabilizing composition;
step 2: adding the nanoparticles that assist in lowering blood lipids and promoting metabolism into the antioxidant stabilizing composition prepared in step 1, mixing them evenly and then dispersing them in the nutritious Camellia diacylglycerol oil mixture to obtain the auxiliary lipid-lowering Camellia diacylglycerol edible oil.
